(19) United States
(12) Patent Application Publication
(10) Pub. No.: US 2015/0320922 A1
(43) Pub. Date: Nov. 12, 2015

(54) METHOD OF PRODUCING A UREA ADSORBENT

(71) Applicant: TRIOMED AB, Lund (SE)

(72) Inventors: Carin MALMBORG, Höör (SE); Nina MEINANDER, Södra Sandby (SE)

(73) Assignee: TRIOMED AB, Lund (SE)

(21) Appl. No.: 14/410,520

(22) PCT Filed: Jul. 5, 2013
(86) PCT No.: PCT/SE2013/000109

§ 371 Date: Dec. 22, 2014

Related U.S. Application Data
(60) Provisional application No. 61/742,659, filed on Aug. 16, 2012.

(30) Foreign Application Priority Data
Jul. 6, 2012 (SE) 1230073-7

(51) Int. Cl. A61M 1/28; B01J 20/10; C08J 9/36; B01J 20/30; C08B 37/08; B01J 20/22; B01J 20/28

(52) U.S. Cl. CPC A61M 1/287; B01J 20/226; B01J 20/103; B01J 20/28033; B01J 20/3071; C08B 37/003; C08J 9/36; C08J 2305/08; C08J 2205/044; C08J 2207/10

(57) ABSTRACT

A method of producing a copper-chitosan polymer material for adsorption of urea from a dialysis solution. A solid chitosan polymer material is immersed in a copper salt solution of a weak acid, such as copper acetate, for allowing the copper ions to complex with the chitosan polymer. The macroporous chitosan polymer membrane has a thickness of no more than about 200 μπι and has a pore size of between about 1 to 100 μπι, for example 20 to 50 μπι. Alternatively, solid chitosan fibers, solid chitosan particles or a chitosan gel bead can be complexed with copper acetate. The concentration of the copper acetate solution is above 50 mM whereby gel formation of the solid chitosan material is avoided during the complexation step.

### FIELD OF INVENTION

The present invention relates to a method of producing a urea adsorbent for use in treatment of renal diseases.

### BACKGROUND

Patients suffering from renal diseases need dialysis treatment. During dialysis, large quantities of dialysate are consumed. The spent dialysate is normally discarded.

In order to reduce the amount of used liquid, the spent dialysis liquid may be regenerated. One previously used method is to pass the spent dialysate through a column comprising urease, which converts urea into ammonia and carbon dioxide. The ammonia is removed by for example zirconium phosphate, since ammonia may be toxic to the patient. However, the conversion of urea by urease and removal of ammonia are difficult to achieve and other methods of removing urea are highly desired.

The spent dialysate of hemodialysis as well as the spent dialysate of peritoneal dialysis may be the subject of such regeneration.

WO 2010/141949 discloses a polymer that interacts or reacts with aqueous urea to aid in regeneration of a dialysate liquid. The polymer may include one or more specific functional groups bonded thereto. Such specific functional groups are selected from carboxylic acid, carboxylic acid esters, carboxylates, amides, dicarboxylic acids, dicarboxylic acid esters and dicarboxylates.

Another promising substance for removing urea from body-fluids is copper-chitosan, as suggested in an article: “Preparation and Characterization of Chitosan/Cu(II) Affinity Membrane for Urea Adsorption”, by Jiahao Liu, Xin Chen, Zhengzhong Shao, Ping Zhou, published in Journal of Applied Polymer Science, Vol. 90, 1108-1112 (2003). A urea adsorption of up to about 80 mg urea per gram chitosan was reported.

In said article, a macroporous chitosan membrane is contacted with copper sulphate in order to form a complex. However, it is difficult to reproduce the data of said article in an industrial scale.

Thus, there is a need in the art for a method of producing a copper-chitosan-membrane, which is industrially reproducible and provides a urea adsorbent having sufficient urea adsorption capacity. The urea amount, which is produced by the body and should be removed by dialysis, is about 15 to 60 g per day.

### SUMMARY OF THE INVENTION

Accordingly, an object of the present invention is to mitigate, alleviate or eliminate one or more of the above-identified deficiencies and disadvantages singly or in any combination.

In an aspect, there is provided a method of producing a copper-chitosan polymer material for regeneration of a dialysis fluid, comprising: providing a solid chitosan polymer material; immersing said solid chitosan polymer material in a copper salt of a weak acid, which is soluble in water, such as: copper acetate, copper formate, copper citrate, copper lactate, copper oxalate, copper propionate, copper benzoate, copper succinate, copper malonate and copper stearate; and allowing the copper ions to complex with the chitosan polymer material during a predetermined incubation time duration, such as between 1 to 24 hours, for example during 2 to 8 hours, for producing said copper-chitosan polymer material. The method may further comprise: rinsing said copper-chitosan polymer material in water after complexation with copper ions, until there are no traces of copper ions. The method may further comprise: drying the rinsed copper-chitosan polymer material.

The solid chitosan polymer material may be a macroporous chitosan polymer membrane, having a thickness of no more than about 200 μm. The macroporous chitosan polymer membrane may have pores of a size between about 1 to 100 μm, for example 20 to 50 μm. The macroporous chitosan polymer membrane may be further grinded in order to form macroporous chitosan polymer particles.

In an embodiment, the macroporous chitosan polymer membrane may be produced by: mixing silica particles with a chitosan polymer solution under heavy stirring until the silica particles are evenly distributed and coated with the chitosan polymer solution; drying the chitosan-silica mixture in thin sheets for forming chitosan polymer membranes; adding sodium hydroxide for dissolving the silica particles for forming a macroporous chitosan polymer membrane; and washing the macroporous chitosan polymer membrane in water to neutral pH. The silica particles may have a size in the range of 5 to 50 μm, for example 5 to 40 μm.

In another embodiment, the solid chitosan polymer material may be in the form of fibers. The fibers may have a thickness of between 5 and 20 μm, for example between 10 and 15 μm.

In a further embodiment, the solid chitosan polymer material is in the form of gel beads.

In a still further embodiment, the copper salt may be copper acetate solution having a concentration, which is above 50 mM. The concentration may be between 50 mM and 350 mM.

In a yet further embodiment, the method may comprise a conditioning step before the immersing step, wherein said solid chitosan polymer material may be allowed to soak water until it is substantially wetted.

In a further aspect, there is provided a use of a copper-chitosan polymer material produced as defined above for adsorption of urea from a dialysis fluid or a body fluid.

### BRIEF DESCRIPTION OF THE DRAWINGS

Further objects, features and advantages of the invention will become apparent from the following detailed description of embodiments of the invention with reference to the drawings, in which:

FIG. 1 is a structure scheme of chitosan molecules in complex with copper, which can adsorb urea.

### DETAILED DESCRIPTION OF EMBODIMENTS

Below, several embodiments of the invention will be described. These embodiments are described in illustrating purpose in order to enable a skilled person to carry out the invention and to disclose the best mode. However, such embodiments do not limit the scope of the invention. Moreover, certain combinations of features are shown and discussed. However, other combinations of the different features are possible within the scope of the invention.

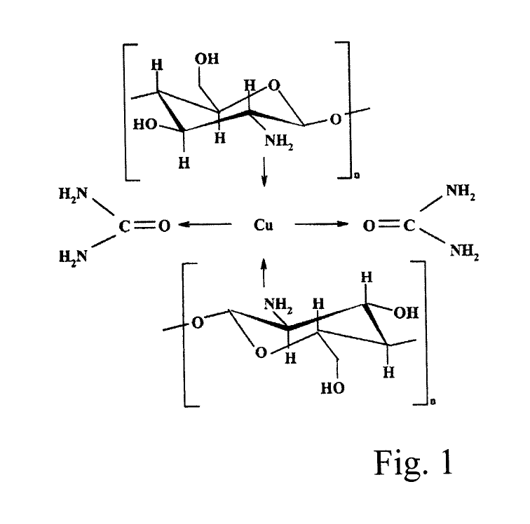

In an embodiment of the invention, there is produced a porous copper-chitosan polymer membrane, wherein copper is complexed to the chitosan polymer amine groups as shown in FIG. 1.

Each copper ion can complex with one or preferably two amine groups of the chitosan polymer, and when two groups from separate chitosan polymer chains are bound by copper, cross-linking and stabilization of the porous membrane is achieved.

The copper-chitosan can readily coordinate with the oxygen of molecules such as urea, and can thus be used as an adsorber of urea, with applications in urea removal from a body fluid, such as blood, plasma, interstitial fluid and urine, and dialysis fluids, such as hemodialysis fluid and peritoneal dialysis fluid.

Chitosan polymer can be provided by different methods. One method of providing a macroporous chitosan membrane has been described in literature, see Ind. Eng. Chem. Res. 1996, 35, 4169-4175, “Control of Pore Sizes in Macroporous Chitosan and Chitin Membranes” by Xianfang Zeng and Eli Ruckenstein.

The pore size of the macropores can be controlled by the size of a porogen, which may be silica particles, which are mixed into a solution of chitosan polymer. The silica particle size may be in the range of 5-50 μm, preferably 5-40 μm. The resulting pores may be in the range of 1-100 μm, preferably 20-50 μm.

Chitosan polymer was dissolved in acetic acid solution and silica particles were added followed by heavy stirring to get the silica particles evenly dispersed and completely coated with chitosan polymer solution. Thereafter, the chitosan polymer was casted into a silica-chitosan membrane and dried. The dry membrane may optionally be grinded into small particles. The dried membrane was immersed in NaOH solution to dissolve the silica particles in order to generate a porous membrane (or particles). Finally, the product was washed to neutral pH.

Another porogen used in the literature is sucrose.

Chitosan of differing molecular weight may be obtained from Primex (Iceland) and Surindo Biotech (Indonesia) in the form of particles and from Hismer Biotechnology (China) in the form of fibers.

Chitosan of four different molecular weights from Primex were used, corresponding to viscosities of 68 (code 43010), 505 (code 43020), 1200 (code 43030), 1215 (code 43040) and 1300 mPa·s (code 43012). The degree of deacetylation was more than 95% for all batches. The particle size was less than 1.5 mm.

Chitosan from Surindo Biotech had a viscosity of 23.8 mPa·s, a degree of deacetylation of 93.6% and a particle size between 1 and 5 mm.

Medical grade chitosan fibers were obtained from Hismer Biotechnology (China). Two different batches of fibers were used, one with a longer fiber length around 20-30 cm, degree of deacetylation 95.3% and the second with a fiber length of about 5 cm, degree of deacetylation of 91.1%. The thicknesses of the fibers were between 5 and 20 μm, mostly between 10 and 15 μm in both batches.

Chitosan gel beads were prepared as described in literature. A chitosan polymer was dissolved in 1-2% acetic acid and the solution was pumped through a hypodermic needle or a glass pipette to form droplets of the chitosan solution, dripping into a bath of 2-5% sodium hydroxide solution. In the high pH of the bath, the droplets solidify into chitosan gel-beads. The beads are kept in the bath for 8-16 hours with slow stirring, and then washed with water until neutral.

Solid chitosan polymer material in the form of particles, macroporous membranes, macroporous particles (ground membranes), fibers or gel beads was treated with copper ions to form a complex of copper with the amine groups of chitosan (FIG. 1).

The complexation process is performed by immersing said solid chitosan polymer material in a solution comprising copper ions.

After the complexation step, the resulting material is rinsed in water during at least 30 minutes. Alternatively, the rinsing step takes place until there are no traces of copper ions in the rinsing solution. The rinsing may take place until the concentration of copper ions is below 0.1 ppm.

The copper ions can be supplied as a soluble copper salt, which is dissolved in water.

The copper salt may be an inorganic salt, including but not limited to copper sulphate, copper chloride, copper bromide, or copper fluoride.

The concentration of such inorganic copper salt solution is normally in the range of 5 to 40 mM, to allow for a sufficient immersion volume for the membrane at stoichiometric or greater amounts of copper ions. The stoichiometric amount of copper ions is equal to half the molar amounts of chitosan monomer units.

The complexation reaction is a sensitive process requiring optimal pH conditions. At too low pH the amine groups are protonated and will not complex with copper ions. At too high pH, insoluble copper substances, such as hydroxides and oxides are easily formed, removing the copper ions from solution.

The copper-amine complex formation is a slow reaction, requiring 1-24 hours, preferably 2-16 hours incubation time. The color of the wet chitosan-copper complex is deep blue, indicating the presence of copper-amine complexes. After drying, the color is blue-grayish.

However, it is difficult to obtain reproducible results, when the copper salt of a strong acid (f.ex. CuSO₄ or CuCl₂) is used. Unexpectedly, it has been found that the use of copper salts of a weak acid provides stable and reproducible results. This may be due to the higher pH of solutions of copper salts of weak acids, than solutions of copper salts of strong acids, favoring the complex formation.

Copper salts of weak acids, which is soluble in water, include but are not limited to copper formate, copper acetate, copper citrate, copper lactate, copper oxalate, copper propionate, copper benzoate, copper succinate, copper maleate and copper stearate, copper acetate being preferred.

The molar amount of copper ions may be equal to or greater than ½ molar amounts of chitosan monomer units.

When a copper salt of an acid, which in its protonated form is a solvent of chitosan (f. ex. copper acetate), is used for copper-chitosan complex formation, it was found that under certain conditions the solid chitosan material is extensively hydrated, forming a gel-like structure. Although we do not want to be bound by a specific explanation, we believe that such hydration may be due to protons released from the chitosan amine groups during contact with a copper salt, forming the corresponding protonated acid. Acid concentrations may vary and locally attain high values, when the acid is entrapped within the solid chitosan structure, such as a porous membrane or fibers located close together.

Such gel-like structure may appear as gel-filled blisters on copper-chitosan porous membranes, or as gel clumps among copper-chitosan fibers, making it difficult to press out liquid from the fibers and thus increasing the wet weight of the material pressed by hand.

Dissolution and gel formation is a drawback in the use of copper-chitosan as a urea adsorbent in medical applications and in flow systems, as dissolved chitosan may contaminate the fluid passing through the adsorbent, and chitosan gel may cause clogging of flow lines and filters in a medical device. In addition, gel formation of an adsorption material may disturb the flow pattern of fluid passing through the material, since preferential flow paths may form, so that the flow of fluid to be regenerated does not contact a large portion of the material.

Unexpectedly, it was found that gel formation during complexation with copper acetate occurred predominantly at low concentrations of copper acetate (5-20 mM), but not at high concentrations (50-350 mM).

When complexing copper onto a porous chitosan membrane, a thin membrane may be used, preferably of a thickness of below about 200 μm. Alternatively the membrane is ground into small particles. This is to avoid entrapment of the acid corresponding to the copper salt used as a source of copper ions, within the membrane structure. Such entrapment is believed to cause gel formation.

The amount of copper ion solution should be sufficient for fully immersing the solid chitosan polymer material during the complexation process. Since the solid chitosan polymer material is hydrohpilic, the material will initially absorb some solution and the amount of copper ion solution should be sufficient for still covering the solid chitosan polymer material after such absorption.

The solid chitosan polymer material (fibers and powders) is normally supplied in a dried form. The solid chitosan polymer material is hydrophilic and may have absorbed different amounts of water in the form of moisture from the surrounding air. In order to condition the solid chitosan polymer material, it may be allowed to soak water for absorption of water before being immersed in the copper ion solution for the complexation process. Such soaking may take place during from 5 to 30 minutes. The conditioning or soaking may take place by placing the material in a humid environment or by exposing the material for a mist or droplets of water.

Alternatively, the soaking is performed by immersing the material in water until it is substantially wetted and cannot adsorb any further water, whereupon surplus water is removed. The soaking of the solid chitosan polymer material in water is expected to facilitate uniform distribution of the copper ions during the following immersion step.

After the soaking step, there may be a relaxation step of at least 5 minutes, for example at least 30 minutes.

In addition to water, the soaking solution may comprise copper acetate, copper formate, copper citrate, copper lactate, copper oxalate, copper propionate, copper benzoate, copper succinate, copper malonate and copper stearate, or any combination of such copper salts. If copper acetate is used, the concentartion should be above 50 mM.

### Example 1

Macroporous chitosan membrane was prepared as described in literature from Chitoclear 43012. 8.5 g macroporous chitosan membranes were incubated in 300 ml of 50 mM CuSO₄ solution for 19 hours on an orbital shaker at room temperature. The membranes were washed with distilled water. The membranes were incubated in 80 ml of 22 mM urea in a peritoneal dialysis fluid during 16 hours on an orbital shaker at room temperature. The amount of urea bound to the membrane was determined to be 4.7 mg urea/g dry chitosan membrane, by analyzing the urea content of the solution before and after incubation.

### Example 2

Macroporous chitosan membrane was prepared as described in literature from Chitoclear 43012. 0.5 g macroporous chitosan membranes were added to 80 ml of 20 mM Cu-acetate solution and incubated on an orbital shaker for 16 hours at room temperature. The membranes were washed with distilled water. The membranes were incubated with 115 ml of a 22 mM urea solution in peritoneal dialysis fluid during 5 hours on an orbital shaker at room temperature. The amount of urea bound to the membrane was determined to be 27 mg urea/g dry chitosan membrane, by analyzing the urea content of the solution before and after incubation.

### Example 3

Macroporous chitosan membrane was prepared as described in literature from Chitoclear 43012. The membrane was cast at a lower thickness compared to Example 3. 0.07 g macroporous chitosan membranes were added to 19 ml of 20 mM Cu-acetate solution and incubated on an orbital shaker for 8 hours at room temperature. The membranes were washed with distilled water. The membranes were incubated with 5 ml of a 22 mM urea solution in peritoneal dialysis fluid during 5 hours on an orbital shaker at room temperature. The amount of urea bound to the membrane was determined to be 35 mg urea/g dry chitosan membrane, by analyzing the urea content of the solution before and after incubation.

### Example 4

Macroporous chitosan membrane was prepared as described in literature from Chitoclear 43012. 0.5 g macroporous chitosan membranes were incubated in 250 ml of 50 mM copper sulphate solution for 3.5 hours on an orbital shaker at room temperature. The membranes were washed with distilled water. The membranes were incubated in 80 ml of 22 mM urea in peritoneal dialysis salt solution during 2 hours on an orbital shaker at room temperature. The amount of urea bound to the membrane was determined to be 10 mg urea/g dry chitosan membrane, by analyzing the urea content of the solution before and after incubation.

### Example 5

Macroporous chitosan membrane was prepared as described in literature from Chitoclear 43012. 1.0 g macroporous chitosan membranes were incubated in 250 ml of 50 mM copper chloride solution for 3.5 hours on an orbital shaker at room temperature. The membranes were washed with distilled water. The membranes were incubated in 80 ml of 22 mM urea in peritoneal dialysis salt solution during 2 hours on an orbital shaker at room temperature. The amount of urea bound to the membrane was determined to be 12 mg urea/g dry chitosan membrane, by analyzing the urea content of the solution before and after incubation.

### Example 6

Macroporous chitosan membrane was prepared as described in literature from Chitoclear 43012. 2.5 g macroporous chitosan membranes were incubated in 1 L of 5 mM copper acetate solution for 3.5 hours on an orbital shaker at room temperature. The membranes were washed with distilled water. The membranes were incubated in 80 ml of 22 mM urea in peritoneal dialysis salt solution during 2 hours on an orbital shaker at room temperature. The amount of urea bound to the membrane was determined to be 15 mg urea/g dry chitosan membrane, by analyzing the urea content of the solution before and after incubation.

### Example 7

1.9 g chitosan Hismer fibers were incubated in 1000 ml of 5 mM copper sulphate solution for 16 hours on an orbital shaker at room temperature. The fibers were washed with distilled water. The fibers were incubated in 140 ml of 12 mM urea in peritoneal dialysis salt solution during 2 hours on an orbital shaker at room temperature. The amount of urea bound to the fibers was determined to be 10 mg urea/g dry chitosan fibers, by analyzing the urea content of the solution before and after incubation.

### Example 8

1.5 g chitosan Hismer fibers were incubated in 1000 ml of 5 mM copper chloride solution for 16 hours on an orbital shaker at room temperature. The fibers were washed with distilled water. The fibers were incubated in 140 ml of 12 mM urea in peritoneal dialysis fluid during 2 hours on an orbital shaker at room temperature. The amount of urea bound to the fibers was determined to be 13 mg urea/g dry chitosan fibers, by analyzing the urea content of the solution before and after incubation.

### Example 9

1.5 g chitosan Hismer fibers were incubated in 1000 ml of 5 mM copper acetate solution for 16 hours on an orbital shaker at room temperature. The fibers were washed with distilled water. The fibers were incubated in 140 ml of 12 mM urea in peritoneal dialysis salt solution during 2 hours on an orbital shaker at room temperature. The amount of urea bound to the fibers was determined to be 20 mg urea/g dry chitosan fibers, by analyzing the urea content of the solution before and after incubation.

### Example 10

3.0 g chitosan gel beads prepared as describes in the literature from Chitoclear 43010 and then homogenized into smaller particles, were incubated in a volume enough to cover the gel beads of 20 mM copper acetate solution. The gel beads were washed with distilled water, and then incubated in 250 ml 12 mM urea in peritoneal dialysis salt solution during 2 hours on an orbital shaker at room temperature. The amount of urea bound to the gel beads was determined to be 29 mg urea/g dry chitosan gel beads, by analyzing the urea content of the solution before and after incubation.

### Example 11

Eight samples of a 5.0 g Hismer fibers were soaked in distilled water for 16 hours, the wet weight was between 6.4-6.8 g for all samples. Eight different copper solutions were prepared from copper acetate between 0-350 mM, table 1. Each sample was soaked in 200 ml copper solution during 24 hours, and a qualitative assessment was made to investigate if gel was formed in the samples. The weight was measured before and after copper treatment. Not only the qualitative assessment, but also the weight of the samples shows in which samples gel was formed, as the gel holds more water than solid fibers.

TABLE 1

| copper acetate | wet weight after | gel formation |
|---|---|---|
| concentration (mM) | copper treatment (g) | (qualitative assessment) |
| 0 | 6.4 | no gel |
| 5 | 88 | gel |
| 10 | 115 | gel |
| 20 | 75 | gel |
| 50 | 20 | no gel |
| 100 | 17 | no gel |
| 200 | 15 | no gel |
| 350 | 15 | no gel |

Since copper acetate has a saturation concentration of about 400 mM, it is expected that no gel is formed for all concentrations above 50 mM and up to the saturation concentration.

The same experiments as indicated above for copper acetate, may be performed by exchanging copper acetate with copper formate, copper citrate, copper lactate, copper oxalate, copper propionate, copper benzoate, copper succinate, copper malonate or copper stearate, or any combinations thereof. It is expected that such experiments will yield the same or similar results.

It is mentioned, that the amount of urea adsorbed to the membrane or fibers or gel beads is more or less independent of such gel formation. However, gel formation will be disadvantageous when considering the final use of the adsorbent material in an adsorbent column, of several reasons, as mentioned above. In addition, gel formation will destroy the macroporous character of a macroporous membrane.

In the examples, a fluid with the same composition as commercial peritoneal dialysis fluid was used in the urea binding step, in order to mimic the conditions (pH, ionic strength etc.) of a peritoneal dialysis fluid regeneration system. The urea concentrations of 12 mM or 22 mM was chosen based on data on urea concentrations in spent peritoneal dialysis fluids from patients.

Binding of the copper ions to chitosan in stoichiometric amounts and in the form of the correct amine-copper-complex is of importance for achieving maximal urea binding. Correct and adequate binding is difficult to achieve using inorganic, strong acid-based copper salts, as indicated by the lower urea binding capacity of chitosan complexed with CuSO₄ or CuCl₂.

For maximal urea binding, the chitosan complexed copper ions should be exposed to the urea molecules in the solution. A thin chitosan macroporous membrane or small particles may increase the accessibility of copper ions within the material, and therefore give a higher binding of urea. In addition, chitosan-copper solid material in the form of fibers may be used. Moreover, chitosan-copper in the form of gel-beads may also be used.

In the claims, the term “comprises/comprising” does not exclude the presence of other elements or steps. Furthermore, although individually listed, a plurality of means, elements or method steps may be implemented by e.g. a single unit. Additionally, although individual features may be included in different claims or embodiments, these may possibly advantageously be combined, and the inclusion in different claims does not imply that a combination of features is not feasible and/or advantageous. In addition, singular references do not exclude a plurality. The terms “a”, “an”, “first”, “second” etc. do not preclude a plurality. Reference signs in the claims are provided merely as a clarifying example and shall not be construed as limiting the scope of the claims in any way.

Although the present invention has been described above with reference to specific embodiment and experiments, it is not intended to be limited to the specific form set forth herein. Rather, the invention is limited only by the accompanying claims and, other embodiments than those specified above are equally possible within the scope of these appended claims.

1. A method of producing a copper-chitosan polymer material for regeneration of a dialysis fluid, comprising
providing a solid chitosan polymer material;
immersing said solid chitosan polymer material in a copper salt of a weak acid, which is soluble in water, such as: copper acetate, copper formate, copper citrate, copper lactate, copper oxalate, copper propionate, copper benzoate, copper succinate, copper malonate and copper stearate;
allowing the copper ions to complex with the chitosan polymer material during a predetermined incubation time duration, such as between 1 to 24 hours, for example during 2 to 8 hours, for producing said copper-chitosan polymer material.

2. The method according to claim 1, further comprising rinsing said copper-chitosan polymer material in water after complexation with copper ions, until there are no traces of copper ions.

3. The method according to claim 2, further comprising drying the rinsed copper-chitosan polymer material.

4. The method according to claim 1,
wherein the solid chitosan polymer material is a macroporous chitosan polymer membrane, having a thickness of no more than about 200 μm.

5. The method according to claim 4, wherein said macroporous chitosan polymer membrane has pores of a size between about 1 to 100 μm.

6. The method according to claim 4, wherein said macroporous chitosan polymer membrane has pores of a size between about 20 to 50 μm.

7. The method according to claim 4, wherein the macroporous chitosan polymer membrane is further grinded in order to form macroporous chitosan polymer particles.

8. The method according to claim 4, wherein said macroporous chitosan polymer membrane is produced by:
mixing silica particles with a chitosan polymer solution under heavy stirring until the silica particles are evenly distributed and coated with the chitosan polymer solution;
drying the chitosan-silica mixture in thin sheets for forming chitosan polymer membranes;
adding sodium hydroxide for dissolving the silica particles for forming a macroporous chitosan polymer membrane; and
washing the macroporous chitosan polymer membrane in water to neutral pH.

9. The method according to claim 8, wherein the silica particles have a size in the range of 5 to 50 μm.

10. The method according to claim 8, wherein the silica particles have a size in the range of 5 to 40 μm.

11. The method according to claim 1, wherein the solid chitosan polymer material is in the form of fibers.

12. The method according to claim 11, wherein the fibers have a thickness of between 5 and 20 μm.

13. The method according to claim 11, wherein the fibers have a thickness of between 10 and 15 μm.

14. The method according to claim 1, wherein the solid chitosan polymer material is in the form of gel beads.

15. The method according to claim 1, wherein said copper salt is copper acetate solution having a concentration, which is above 50 mM.

16. The method according to claim 15, wherein said copper acetate solution has a concentration, which is between 50 mM and 350 mM.

17. The method according to claim 1, further comprising a conditioning step before the immersing step, wherein said solid chitosan polymer material is allowed to soak water until it is substantially wetted.

18. A method of using a copper-chitosan polymer material produced according to claim 1 for adsorption of urea from a dialysis fluid or a body fluid.
